(19) United States
(12) Patent Application Publication
(10) Pub. No.: US 2012/0064174 A1
(43) Pub. Date: Mar. 15, 2012

(54) METHOD FOR STIMULATING HAIR GROWTH

(75) Inventors: Saad Harti, Lutry (CH); André Ulmann, Paris (FR)

(73) Assignee: LEGACY HEALTHCARE HOLDING LTD., Valleta (MT)

(21) Appl. No.: 13/196,217

(22) Filed: Aug. 2, 2011

Related U.S. Application Data
(60) Provisional application No. 61/370,926, filed on Aug. 5, 2010.

(51) Int. Cl. A61K 33/30; A61P 17/00; A61K 36/752

(52) U.S. Cl. 424/641; 424/736

(57) ABSTRACT

The present teachings provide methods for stimulating the hair growth on a human head including simultaneous administration:
a) by oral or transdermal route of a composition containing a 5-alpha-reductase inhibitor or a peripheral vasodilator, and
b) by topical route of a composition containing as active ingredient an extract of Allium species, an extract of Citrus species and
   either an extract of Paullinia species and an extract of Theobroma species
   or an extract of Salix species and zinc sulfate.

### CROSS-RELATED APPLICATIONS

This application claims the benefit of U.S. Provisional Application Ser. No. 61/370,926, filed on Aug. 5, 2010, which is incorporated by reference herein.

### FIELD

The present teachings provide methods for stimulating hair growth.

### BACKGROUND

Hair loss and hair thinning are sensitive issues for patients having an undesired reduction in their hair volume. Treatments for hair loss and hair thinning may provide limited results. Various systems have been introduced to address hair loss and hair thinning, such as surgical intervention, cosmetic devices, and therapeutic treatments.

An exemplary therapeutic treatment is administration of a 5-alpha-reductase inhibitor to effect hair growth. Yet another therapeutic treatment is administration of a peripheral vasodilator to effect hair growth. Another exemplary therapeutic treatment is provided in WO 2008/113912 that details how compositions containing an extract of Allium species; an extract of Citrus species; and either an extract of Paullinia species and an extract of Theobroma species or an extract of Salix species, and zinc sulfate as active ingredients increase the hair growth.

Despite these and other treatments, patients still desire rapid results to increase hair thickness and to reverse or prevent hair loss.

### SUMMARY

In various aspects, the present teachings provide methods for stimulating hair growth on a human head by simultaneously administering:

a) by oral or transdermal route of a composition containing a 5-alpha-reductase inhibitor or a peripheral vasodilator, and

b) by topical route of a composition containing as active ingredient an extract of Allium species; an extract of Citrus species; and either:
i) an extract of Paullinia species and an extract of Theobroma species, or
ii) an extract of Salix species and zinc sulfate.

In still other aspects, the methods for stimulating hair growth on a human head include:

a) administering an oral or transdermic composition containing a 5-alpha-reductase inhibitor or a peripheral vasodilator for a treatment period of at least three months; and

b) topically applying a lotion during the treatment period, where the lotion comprises an extract of Allium species, an extract of Citrus species and either:
i) an extract of Paullinia species and an extract of Theobroma species, or
ii) an extract of Salix species and zinc sulfate.

In further aspects, the methods for stimulating hair growth on a human head having hair of an initial diameter thickness include:

a) administering to the head an oral or transdermic composition containing a 5-alpha-reductase inhibitor or a peripheral vasodilator for a treatment period of at least three months;

b) topically applying a combination of Allium cepa, Citrus limon, Salix alba, and zinc sulfate during the treatment period; and

c) increasing the diameter of hair to a post-treatment period diameter that is larger than the initial diameter to thicken the hair.

### DETAILED DESCRIPTION

The present teachings provide methods for increasing hair growth. The treatment method is a combination of several therapies that provides an unexpected and synergistic result. As used herein, “synergy” or “synergistic” refers to the therapies being combined such that the therapeutic effect of simultaneous administration is greater than the additive effect of administering the respective therapies separately.

It has been discovered that the simultaneous administration:

a) by oral or transdermal route of a composition containing a 5-alpha-reductase inhibitor or a peripheral vasodilator, and

b) by topical route of a composition containing as active ingredient an extract of Allium species; an extract of Citrus species; and either:
i) an extract of Paullinia species and an extract of Theobroma species, or
ii) an extract of Salix species and zinc sulfate has a novel and enhanced effect on the hair growth compared to the single treatments alone.

Thus the present teachings concern the simultaneous administration:

a) by oral or transdermal route of a composition containing a 5-alpha-reductase inhibitor or a peripheral vasodilator, and

b) by topical route of a composition containing as active ingredient an extract of Allium species, an extract of Citrus species and either
i) either an extract of Paullinia species and an extract of Theobroma species, or
ii) an extract of Salix species and zinc sulfate.

Among the methods of the present teachings, those which are of interest are the methods in which the 5-alpha-reductase inhibitor is selected from finasteride and dutasteride. Of particular interest are methods using finasteride as the 5-alpha-reductase inhibitor.

The peripheral vasodilators are selected from minoxidil and aminexil. Aminexil is of particular interest in methods of the present teachings.

The preferred composition for topical administration contains from 65% to 93% of an aqueous-alcoholic extract of Allium species, from 5% to 33% of an aqueous-alcoholic extract of Citrus species, from 0.25% to 2.5% of an aqueous alcoholic extract (atomized or non-atomized) of Paullinia species, and from 0.25% to 2.5% of an aqueous-alcoholic extract (atomized or non-atomized) of Theobroma species.

In yet other aspects, more preferred compositions are those containing from 65% to 93% of an aqueous-alcoholic extract of Allium species, from 5% to 33% of an aqueous-alcoholic extract of Citrus species, from 0.25% to 2.5% of an aqueous-alcoholic extract (atomized or non-atomized) of Paullinia species and from 0.25% to 2.5% of an aqueous-alcoholic extract (atomized or non-atomized) of Theobroma species and especially those containing from 65% to 93% of an aqueous-alcoholic extract of Allium cepa, from 5% to 33% of an aqueous-alcoholic extract of Citrus limon, from 0.25% to 2.5% of an aqueous-alcoholic extract (atomized or non-atomized) of Paullinia species and from 0.25% to 2.5% of an aqueous-alcoholic extract (atomized or non-atomized) of Theobroma species.

The term extract of Allium species or aqueous-alcoholic extract of Allium species refers particularly to aqueous-alcoholic extracts and native extracts obtained from all species of the genus Allium (family Liliaceae) and especially Allium cepa. Extract of Citrus species or aqueous-alcoholic extract of Citrus species refers particularly to aqueous-alcoholic extracts and native extracts obtained from all species of the genus Citrus (family Rutaceae) and especially Citrus limon. Extract (atomized or non-atomized) of Paullinia species or aqueous-alcoholic extract (atomized or non-atomized) of Paullinia species refers particularly to aqueous-alcoholic extracts and native extracts obtained from all species of the genus Paullinia (family Sapindaceae) and especially Paullinia cupana. Extract (atomized or non-atomized) of Theobroma species or aqueous-alcoholic extract (atomized or non-atomized) of Theobroma species refers particularly to aqueous-alcoholic extracts and native extracts obtained from all species of the genus Theobroma (family Malvaceae) and especially Theobroma cacao.

Among the compositions according to the present teachings, those which are also of most particular interest are compositions as defined above containing from 65% to 93% of an aqueous-alcoholic extract of Allium species, from 5% to 33% of an aqueous-alcoholic extract of Citrus species, from 0.25% to 2.5% of an aqueous extract (atomized or non-atomized) of Salix species, and from 0.1% to 1% of zinc sulfate. Of additional interest are compositions containing from 65% to 93% of an aqueous-alcoholic extract of Allium cepa, from 5% to 33% of an aqueous-alcoholic extract of Citrus limon, from 0.25% to 2.5% of an aqueous extract (atomized or non-atomized) of Salix species and from 0.1% to 1% zinc sulfate hexahydrate.

Extract of Salix species or aqueous extract (atomized or non-atomized) of Salix species refers to aqueous extracts obtained from all species of the genus Salix (family Salicaceae), especially Salix alba. The zinc sulfate used in compositions according to the present teachings may be in the form of the anhydrous salt or a polyhydrated salt, especially the hexahydrate.

The most preferred compositions used according to the present teachings include those containing approximately 87% of an aqueous-alcoholic extract of Allium cepa, approximately 12% of an aqueous-alcoholic extract of Citrus limon, approximately 0.5% of an aqueous-alcoholic extract (atomized or non-atomized) of Paullinia cupana, and approximately 0.5% of an aqueous-alcoholic extract (atomized or non-atomized) of Theobroma cacao. Other preferred compositions include those containing approximately 87% of an aqueous-alcoholic extract of Allium cepa, approximately 12% of an aqueous-alcoholic extract of Citrus limon, approximately 0.5% of an aqueous extract (atomized or non-atomized) of Salix alba, and 0.2% zinc sulfate hexahydrate.

These compositions are prepared as indicated in patent application WO 2008/113912, which is incorporated in its entirety by reference. These pharmaceutical compositions are prepared by conventional methods, in which pharmaceutically inert, organic or inorganic excipients are added to the compositions obtained according to the present teachings.

According to the present teachings the 5-alpha-reductase inhibitor or the peripheral vasodilator is administered daily during a period of several months with a composition containing as active ingredient an extract of Allium species, an extract of Citrus species, and either an extract of Paullinia species and an extract of Theobroma species or an extract of Salix species and zinc sulfate.

The 5-alpha-reductase inhibitor or the peripheral vasodilator is used according to conventional medicine and conventional administration techniques of the respective compositions. They are administered daily by oral route at a dose of 0.5 mg to 5 mg. As a non-limiting example, finasteride is administered daily at a dose of 1 mg by oral route.

In order to obtain optimal results for hair growth, the administration of the 5-alpha-reductase inhibitor or the peripheral vasodilator along with the combination of extracts is conducted over a period of at least three months. When using the compositions obtained according to the present teachings, doses may vary within relatively wide limits and must be set according to the person being treated and the condition concerned. Pharmaceutical compositions normally contain from 0.2 to 500 mg, preferably from 1 to 200 mg, of active ingredients as defined above, in the form of dry extract.

### Example of Treatment

Finasteride, a 5-alpha-reductase inhibitor, was administered during several weeks at a daily dosage of 1 mg (oral route) per day to a group of 4 men. During that period of several weeks, the patients also have a daily application of a scalp a lotion (hereinafter Composition A is Cellium® GC 210 mg/mL) prepared as indicated in example 1 of patent application WO 2008/113912. The lotion contains:

| Ingredient | Amount |
|---|---|
| an aqueous-alcoholic extract of Allium cepa | 87.04% |
| an aqueous-alcoholic extract of Citrus limon | 11.96% |
| an atomized or non-atomized aqueous-alcoholic | 0.50% |
| extract of Paullinia cupana | |
| an atomized or non-atomized aqueous-alcoholic | 0.50% |
| extract of Theobroma cacao | |

After a several weeks' treatment, it has been observed that the group of men receiving simultaneously the treatment of a 5-alpha-reductase inhibitor (finasteride), and composition A had an increase of the growth of their hair in comparison to the group of persons receiving a 5-alpha-reductase inhibitor (finasteride) and placebo on the scalp. A clinical study of the method for stimulating the hair growth on a human scalp has been performed.

### Study Objectives

The aim of the study is to measure, for 12 consecutive months, the clinical efficacy of the anti-hair loss topical solution, Cellium (TRADEMARK) GC 210 mg/mL, versus a Placebo, on the scalp of male volunteers. The male volunteers had been under treatment of a 5-alpha-reductase inhibitor treatment (Finasteride 1 mg) started within the last 3 months, or for the last three years. The first objective is to assess if the hair diameter of volunteers under a 5-alpha-reductase inhibitor (Finasteride 1 mg) and Cellium (TRADEMARK) GC 210 mg/mL is higher than on volunteers under a 5-alpha-reductase inhibitor (Finasteride 1 mg) and placebo only. The hair diameters were compared using a starting or initial hair diameter measurement and a post-study hair diameter measurement as quantified later herein. The second objective is to evaluate the cosmetic acceptance in the studied population.

### Study Rational

Cellium (TRADEMARK) GC 210 mg/mL has a proven efficacy in preventing hair loss and promoting hair growth. A previous clinical study by a phototrichogram method has already proved that Cellium (TRADEMARK) GC 210 mg/mL significantly increases the number of hairs in the anagen phase, significantly decreases the number of hairs in the telogen phase, leading to a normalization of the anagen/telogen ratio after six (6) weeks of treatment only.

Moreover, it has also been proven that Cellium (TRADEMARK) GC when tested on endothelial cells, in vitro, has effect on capillary recruitment.

Based on this background, we expect to get a synergic effect when combining a 5-alpha-reductase inhibitor treatment (Finasteride 1 mg) acting on androgens, and Cellium (TRADEMARK) GC treatment, especially on the diameter of the hair.

### Methodology and Design

A prospective, double-blind, monocentric, randomized testing along with a placebo testing is conducted with two parallel groups. The clinical trial is performed, for 12 consecutive months, on male volunteers having started a 5-alpha-reductase inhibitor treatment (Finasteride 1 mg) within the last 3 months, or for the last three years, with evaluation by diameter measurement of hair and photographic documentation at different times. Information on self-assessed effectiveness, appreciation, and overall satisfaction was solicited from volunteers using a questionnaire.

### Number of Volunteers

The study will present four groups. Group A1 includes 40 male volunteers having started a 5-alpha-reductase inhibitor treatment (Finasteride 1 mg) within the last 3 months plus the Placebo. Group B1 includes 40 male volunteers having started a 5-alpha-reductase inhibitor treatment (Finasteride 1 mg) started within the last 3 months plus Cellium (TRADEMARK) GC. Group A2 includes 40 male volunteers under a 5-alpha-reductase inhibitor treatment (Finasteride 1 mg) for more than three years plus Placebo. Group B2 includes 40 male volunteers under a 5-alpha-reductase inhibitor treatment (Finasteride 1 mg) for more than three years plus Cellium (TRADEMARK) GC.

At least 25 volunteers in each Group are analyzed.

### Inclusion Criteria

The following are inclusion criteria for the study:
Men;
Aged between 20 and 59 years;
Normality of preliminary examination;
Androgenetic alopecia, Hamilton-Norwood's Classification III to IV;
Starting a 5-alpha-reductase inhibitor treatment (Finasteride 1 mg) within the last 3 months or more than 3 years;
Volunteers accepting hair clipping;
Volunteer in good general health;
Volunteer without dermatologic lesions on the scalp;
Volunteer without allergic background to cosmetic products;
Last shampoo 24 hours+1-6 hours before visit;
Neither anti-hair loss treatment in progress nor anti-hair loss treatment stopped within the last 12 months (except a 5-alpha-reductase inhibitor treatment for both cases);
Volunteer agreeing not to amend either his diet or his capillary custom during the study;
Volunteer able to give his informed consent;
Volunteer cooperating, warned of the necessity and duration of controls;
Volunteer able to follow the study protocol until its completion; and
Written informed consent signed.

### Non-Inclusion Criteria

Non-inclusion criteria for the study are:
Known allergy to one of the ingredients of the formula, including caffeine and theobromine present in plant extracts;
Current treatment affecting hair cycle;
Minors, elderly, incapacitated people, congenital alopecia volunteer, iatrogenous or traumatic alopecia, chemotherapy volunteers and other volunteers with secondary alopecia resulting from an underlying condition or therapy;
Hair transplant surgery volunteers;
Concomitant use of any hair loss product (Except a 5-alpha-reductase inhibitor treatment);
Volunteer with a dietary deficiency or symptoms of anemia;
Volunteer having severe or evolving disease, including endocrine disease;
Volunteer having seborrheic dermatitis or psoriasis, or any scalp dermatitis;
Use of hairspray or styling gel since last shampoo;
Regular use or within the 3 months preceding the visit of a good permanent shaping of hair (hair straighteners, permanent);
Diet in progress or stopped within last 4 months;
Use of any hair loss product within the last 12 months (included a 5-alpha-reductase inhibitor treatment if a previous treatment had already been established);
Non-respect of the protocol;
Volunteer already included in another clinical trial; Volunteer undertaking or planning to conduct another test; and/or
No written informed consent signed.

### Exclusion Criteria

Exclusion criteria for the study are:
Anti-hair loss treatment in progress other than the 5-alpha-reductase inhibitor treatment and test product introduced during the study;
New treatment affecting hair cycle;
Hair transplant surgery;
Volunteer with a dietary deficiency or symptoms of anemia;
Severe or evolving disease, including endocrine disease revealed during the study;
Presence of seborrheic dermatitis or psoriasis or any scalp dermatitis;
Last shampoo apart from the delay of 24 hours+/−6 hours before M3, M6, and M12 visits;
Use of hairspray or styling gel since last shampoos preceding M3, M6, and M12 visits;
Regular use or within the 3 months preceding the visit of hair straighteners, permanent;
Modification of usual capillary custom;
Modification of diet since initiation of study;
Participation in another clinical study; and/or
Onset of an allergic reaction to cosmetic products.

### Test Product, Dose and Mode of Administration

The following details the test product, the dose, and the mode of administration to the volunteer(s) or subject(s) included in the study.

A topical solution of Cellium (TRADEMARK) GC 210 mg/mL is administered by spraying over a wet or dry scalp. Ten (10) to fifteen (15) sprays are administered to the scalp. The scalp is then gently massaged after application. This application is repeated every evening prior to going to bed to provide a daily administration. The treatment is continued for a period of twelve (12) months. The reference therapy is through placebo.

### Criteria for Evaluation:

Several levels of criteria are used for evaluation. With respect to efficacy, a primary criteria is the assessment of product efficacy by clipping the hair clipping for a diameter measurement. The diameter assessment was conducted both before the study to obtain an initial diameter and at the conclusion of the study to obtain a post-treatment diameter to thereby quantify the increased in diameter and thus thickness of the hair. Exemplary secondary criteria include photographic documentation for qualitative purposes and also self-assessed effectiveness, appreciation, and overall satisfaction as indicated by a questionnaire given to participants.

### Statistical Methods

Statistical analyses will be performed carrying out either the Paired t-test, the One Way Analysis of Variance, the Kruskal-Wallis test, the Tukey test, and the Dunnett's method using the SigmaStat software (Jandel Corp, CA, United States) for histopathological and immunohistochemical analyses. Significant data will be considered when P<0.05.

I. Study Objectives

The aim of the study is to measure the clinical efficacy, for 12 consecutive months, of the anti hair loss topical solution, Cellium (TRADEMARK) GC 210 mg/mL, vs. Placebo, on the scalp of male volunteers under a 5-alpha-reductase inhibitor treatment (Finasteride 1 mg treatment) started for less than 1 week, or for more than 3 years. The first objective is to assess if the hair diameter of volunteers under a 5-alpha-reductase inhibitor treatment (Finasteride 1 mg) and Cellium (TRADEMARK) GC 210 mg/mL is higher than on volunteers under a 5-alpha-reductase inhibitor treatment (Finasteride 1 mg) and placebo only. The second objective is to evaluate the cosmetic acceptance in the studied population.

A. Identity of Investigational Product(s)

Cellium (TRADEMARK) GC 210 mg/mL contains the following ingredients:
Active ingredient: Cellium (TRADEMARK) GC (21%): (Composition a Above)
   Allium Cepa (Onion) Bulb Extract
   Citrus Medica Limonum (Limon) Fruit Extract
   Paullinia Cupana Seed Extract
   Theobroma Cacao (Cocoa) Seed Extract
Excipients:
   Aqua/Water (QS 16, 50% m/m)
   Alcohol (Ethanol (+/−10%))
   Betaine
   Glycerin
   Polysorbate 20
   Maltodextrin
   Silica
   Parfum/Fragrance

The placebo formula of Cellium (TRADEMARK) GC 210 mg/mL contains the following ingredients:
Aqua/Water (QS 16, 50% m/m)
Alcohol (Ethanol (+/−10%)
Betaine
Glycerin
Polysorbate 20
Maltodextrin
Silica
Parfum/Fragrance

Clinical observations are provided in the following table that shows that co-administration of a composition containing a 5-alpha reductase inhibitor with a composition containing an extract of Allium Cepa (Onion) Bulb Extract, Citrus Medica Limonum (Limon) Fruit Extract, Paullinia Cupana Seed Extract and Theobroma Cacao (Cocoa) Seed Extract has a positive effect for stimulating the hair growth and especially the increase of hair diameter. Notably, the increase in hair diameter is from about 0.8% to about 4.0% or greater.

| | | Diameter | | Diameter | | | Variation | |
|---|---|---|---|---|---|---|---|---|
| | Days of | at | | at | | | of | Variation of |
| | treatment | inclusion | | inclusion | | Months on | diameter | diameter |
| Treatment | (A) | (μm) | Interval | (μm) | Interval | Finasteride | (μm) | (in %) |
| Finasteride + Placebo | 43 | 50.2 | 5.81 | 52.4 | 6.6 | 3 | 2.2 | 4.4% |
| Finasteride + Placebo | 49 | 46.1 | 3.3 | 45.8 | 5.22 | 54 | −0.3 | −0.7% |
| Finasteride + Placebo | 51 | 51.0 | 5.85 | 49.8 | 7.43 | 0 | −1.2 | −2.4% |
| Finasteridel + Cellium | 45 | 44.8 | 2.41 | 46.6 | 4.32 | 45 | 1.8 | 4.0% |
| Finasteridel + Cellium | 32 | 47.7 | 5.3 | 49.2 | 4.77 | 40 | 1.5 | 3.1% |
| Finasteridel + Cellium | 44 | 48.1 | 4.48 | 48.5 | 6.59 | 56 | 0.4 | 0.8% |

What is claimed is:

1. A method for stimulating hair growth on a human head, comprising the simultaneous administration:
a) by oral or transdermic route of a composition containing a 5-alpha-reductase inhibitor or a peripheral vasodilator, and
b) by topical route of a composition containing as active ingredient an extract of Allium species, an extract of Citrus species and:
(i) either an extract of Paullinia species and an extract of Theobroma species, or
(ii) an extract of Salix species and zinc sulfate.

2. A method for stimulating hair growth according to claim 1, wherein the 5-alpha-reductase inhibitor is selected from the group consisting of finasteride and dutasteride.

3. A method for stimulating hair growth according to claim 2, wherein the 5-alpha-reductase inhibitor is finasteride.

4. A method for stimulating hair growth according to claim 1, wherein the peripheral vasodilator is selected from the group consisting of minoxidil and aminexil.

5. A method for stimulating hair growth according to claim 4, wherein the peripheral vasodilator is aminexil.

6. A method for stimulating hair growth according to claim 1, wherein the topical composition comprises an aqueous-alcoholic extract of Allium species, an aqueous-alcoholic extract of Citrus species and:
(i) either an aqueous alcoholic extract of Paullinia species and an aqueous-alcoholic extract of Theobroma species; or
(ii) an aqueous extract of Salix species and zinc sulfate.

7. A method for stimulating hair growth according to claim 1, wherein the topical composition comprises from 65% to 93% of an aqueous-alcoholic extract of Allium species, from 5% to 33% of an aqueous-alcoholic extract of Citrus species, from 0.25% to 2.5% of an aqueous alcoholic extract of Paullinia species and from 0.25% to 2.5% of an aqueous-alcoholic extract of Theobroma species.

8. A method for stimulating hair growth according to claim 1, wherein a 5-alpha-reductase inhibitor or a peripheral vasodilator is administered daily during a period of several months with a composition comprising as active ingredient an extract of Allium species, an extract of Citrus species and:
(i) either an extract of Paullinia species and an extract of Theobroma species; or
(ii) an extract of Salix species and zinc sulfate.

9. A method for stimulating hair growth according to claim 8, wherein the peripheral vasodilator is administrated daily by oral route at a dose of 0.5 mg to 5 mg.

10. A method for stimulating hair growth on a human head, comprising:
a) administering an oral or transdermic composition containing a 5-alpha-reductase inhibitor or a peripheral vasodilator for a treatment period of at least three months; and
b) topically applying a lotion during the treatment period, where the lotion comprises an extract of Allium species, an extract of Citrus species, and either:
(i) an extract of Paullinia species and an extract of Theobroma species, or
(ii) an extract of Salix species and zinc sulfate.

11. A method for stimulating hair growth according to claim 10, wherein the treatment period includes daily administration of at least one of: a) the oral or transdermic composition and b) the topically applied lotion.

12. A method for stimulating hair growth according to claim 10, wherein the 5-alpha-reductase inhibitor is selected from the group consisting of finasteride and dutasteride.

13. A method for stimulating hair growth according to claim 10, wherein the peripheral vasodilator is selected from the group consisting of minoxidil and aminexil.

14. A method for stimulating hair growth according to claim 10, wherein the topical lotion comprises from 65% to 93% of an aqueous-alcoholic extract of Allium species, from 5% to 33% of an aqueous-alcoholic extract of Citrus species, from 0.25% to 2.5% of an aqueous alcoholic extract of Paullinia species, and from 0.25% to 2.5% of an aqueous-alcoholic extract of Theobroma species.

15. A method for stimulating the hair growth on a human head having hair of an initial diameter, comprising:
a) administering to the head an oral or transdermic composition containing a 5-alpha-reductase inhibitor or a peripheral vasodilator for a treatment period of at least three months;
b) topically applying a combination of Allium cepa, Citrus limon, Salix alba, and zinc sulfate during the treatment period; and
c) increasing the diameter of hair to a post-treatment period diameter that is larger than the initial diameter to thicken the hair.

16. A method for stimulating hair growth according to claim 15, wherein the treatment period includes daily administration of at least one of: a) the oral or transdermic composition and b) the topically applied lotion.

17. A method for stimulating hair growth according to claim 15, wherein the 5-alpha-reductase inhibitor is selected from the group consisting of finasteride and dutasteride.

18. A method for stimulating hair growth according to claim 15, wherein the peripheral vasodilator is selected from the group consisting of minoxidil and aminexil.

19. A method for stimulating hair growth according to claim 15, wherein the topical composition comprises from 65% to 93% of an aqueous-alcoholic extract of Allium cepa, from 5% to 33% of an aqueous-alcoholic extract of Citrus limon, from 0.25% to 2.5% of an aqueous alcoholic extract of Paullinia cupana, and from 0.25% to 2.5% of an aqueous-alcoholic extract of Theobroma cacao.

20. A method for stimulating hair growth according to claim 15, wherein the diameter of the hair is increased by up to about 4.0% as compared to the initial thickness.
